(19) United States
(12) Patent Application Publication
(10) Pub. No.: US 2009/0216173 A1
(43) Pub. Date: Aug. 27, 2009

(54) Blood detoxification membrane, method for producing same, and use thereof

(76) Inventors: Jürgen Gensrich, Teltow (DE); Hans-Peter Fink, Teltow (DE); Gerd Weidel, Potsdam (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/596,114

(22) PCT Filed: May 12, 2005
(86) PCT No.: PCT/EP05/05175

§ 371 Date: Dec. 9, 2008

(30) Foreign Application Priority Data
May 12, 2005 (DE) 10 2004 023 410.8

(51) Int. Cl. A61M 1/18; B05D 3/00; B32B 1/08; B32B 1/00; A61M 1/34

(52) U.S. Cl. 604/6.04; 427/2.31; 428/36.92; 428/36.9; 604/6.09

(57) ABSTRACT

The invention relates to membranes for haemodialysis, haemofiltration and/or plasmapheresis formed from substituted or unsubstituted cellulose carbamate having a carbamate nitrogen content in the range between 0.1 and 6%. The invention also relates to a method for manufacturing membranes of this type as well as to their use for blood detoxification within the framework of ultrafiltration, high flux dialysis, haemo-diafiltration and plasmapheresis of the blood.

The invention relates to membranes for haemodialysis, haemofiltration and/or plasmapheresis, formed from substituted or unsubstituted cellulose carbamate having a carbamate nitrogen content in the range between 0.1 and 6%. The invention also relates to a method for manufacturing membranes of this type as well as to their use for blood detoxification within the framework of ultrafiltration, high flux dialysis, haemodia-filtration and plasmapheresis of the blood.

For detoxifying the blood in the case of kidney failure by means of dialysis and ultrafiltration, treatment with artificial kidneys based on polymer membranes is a well-established and successful medical treatment method. Artificial kidneys are known with flat membranes, with tubular membranes and with hollow membranes. The latter are also referred to as hollow fibres or hollow-fibre membranes. Inter alia on account of the low blood replenishment volume, artificial kidneys with hollow membranes, also referred to as hollow-fibre dialysers, are used by preference. Depending on the permeability of the membrane and the requirement for blood detoxification, treatment methods include inter alia dialysis, ultrafiltration of the blood, also haemofiltration, high flux dialysis and haemodiafiltration or also plasma separation or plasmapheresis. Each of these presents particular requirements of the properties of the components of the substance-separating equipment as well as their construction and geometry, especially of the membranes.

The main components of artificial kidneys and blood detoxification equipment—here referred to in short as artificial kidneys—are polymer membranes. They separate the blood from the dialysate and/or the ultrafiltrate and through them the transport from the blood of the substances which have to be excreted through the urine takes place. Their properties in respect of substance transport and haemocompatibility determine the efficiency of the artificial kidneys. Driving forces of the substance transport are the transmembrane pressure difference and/or the concentration difference.

For the manufacture of polymer membranes for artificial kidneys, both synthetic and natural polymers or their derivatives are used as the polymer materials, such as, for example, polysulphones, polyamides or polyacrylonitrile on the one hand and regenerated cellulose or cellulose acetate on the other hand.

The preferred, oldest and most successful polymer membrane material is regenerated cellulose. Exactly like synthetic polymer membranes, membranes formed from regenerated cellulose can be manufactured using completely different independent methods, e.g. according to the Cuoxam process (DE 23 28 853), according to the viscose process (DD 30 17 49) or the amine oxide process (EP 0 807 460). Each of these procedures requires its own independent technical solutions in order to guarantee membrane transport and haemocompatibility in respect of the medical requirements. This also applies to cellulose derivatives, e.g. cellulose acetate, which are generally decomposed into regenerated cellulose before they are used (U.S. Pat. No. 3,546,209).

Whilst the substance transport is determined by the particular cavity structure and the cavity distribution as well as the hydrophilicity of the membrane materials, the haemocompatibility, i.e. e.g. the complement activation and/or the thrombogenicity and thus the capacity for adsorbing body proteins, also depends on the chemical structure of the polymer material and its distribution. These processes are possibly responsible for compatibility in a patient and are still being very much discussed.

All synthetic polymer membranes represent substances which are foreign to the body and there is a number of attempts to introduce recognition patterns for foreign substances in the body through high hydrophilicity, high hydrophobicity, through a combination of both and/or through specific groupings which are bound to the membrane-forming base polymer or also through admixtures to the base polymer or through surface reactions and/or coatings on the blood-wetted side of the polymer.

Membrane materials comprising regenerated cellulose are characterized by a relatively high complement activation (D. E. Chenoweth, Artificial Organs 8(3) (1984) 281; DE 34 30 503). On the other hand, cellulose is naturally hydrophilic and is not characterized by thrombogenicity as much as many synthetic materials. Complement activation is repressed above all by e.g. a whole number of additives such as DEAE cellulose in the Cuoxam process (DE 34 30 503) or for example cellulose sulphate sodium inter alia in the viscose process (DD 299 070). The manufacture of such substances and their processing into the membrane materials having altered properties are complicated and expensive. It would therefore be better to use directly formable cellulose substances which lead immediately to material which is compatible with blood. Furthermore it is complicated and expensive to improve the transport porosity of the membrane material and control it to the desired extent by using specific additives, such as in the viscose process (DD 300 037) for example. It would also be better here to use a cellulose material which provides these properties, can be controlled directly by the process and thus continues in a simple manner the decades-long good experience of the use of cellulose as a membrane material in an artificial kidney for a patient.

Another cellulose which is gaining in importance is cellulose carbamate. The carbamate process is known admittedly for textile purposes (Ekman, K., Eklund, V., Fors, J., Huttunen, J. 1., Selin, J-F and Turunen, O. T.: “Cellulose Carbamate” 1986, New York, edited Young, R. A. and Rowell, R. M. Cellulose: Structure, Modification and Hydrolysis, pp. 131-148; M. Brück, M. Voges, H.-P. Fink, J. Gensrich, F. Hermanutz and F. Gähr; “Der CARBACELL® Prozess, eine umweltfreundliche Alternative zur Herstellung cellulosischer Regeneratfasern” [=“The CARBACELL® process, an environmentally friendly alternative to the manufacture of regenerated cellulose fibres”, lecture by M. Voges at the ZELLCHEMING main assembly of 25-28 Jun. 2001 in Baden-Baden (Conference record), but the production of blood detoxification membranes is not so far known in the prior art.

Up to now it has merely been known that derivatives of cellulose carbamate can be synthesised e.g. in LiCl/DMAc or NMP/LiCl and similar systems and formed from such systems (Diamantouglou, M., Platz, J., Vienken, J.: Artificial Organs V23, Issue 1, January 1999, 15-22) or can be used as a coating material (U.S. Pat. No. 5,360,636) and thus improve haemocompatibility if a quite specific low concentration in the order of magnitude of the average substitution degree of approximately 0.1 on contact with blood is effective. But in both cases there is the regrettable very high expenditure with its separate manufacture and specific treatment. Controlling the porosity by deforming or coating cannot be taken from these publications. The membrane properties thus relate largely to the base polymer for the membrane process and limit these e.g. in the case of a coating, as is generally known

Proceeding from this prior art, the object of the present invention was to provide membranes of which the transport porosity and haemocompatibility can be controlled or improved by the manufacturing process.

This object is accomplished by the method according to the invention having the features of claim 1 and the membrane according to the invention having the features of claim 18. In claim 27 is quoted an inventive use of the membranes. The additional dependent claims list advantageous developments.

According to the invention, a method for the manufacture of membranes for haemodialysis, haemofiltration and/or plasmapheresis is made available, comprising the following method steps:

a) dissolving cellulose carbamate with a carbamate nitrogen content of 1 to 6% in sodium hydroxide solution, followed by shaping into a membrane,

b) coagulation with acid or alkaline solutions,

c) washing the membrane,

d) finishing the membrane by adding a pore-preserving processing aid which penetrates into the pores of the membrane,

e) drying the membrane.

Surprisingly it could be demonstrated that through the use of cellulose carbamate both control of the transport porosity and good haemocompatibility can be achieved, specifically when the membranes and hollow membranes are manufactured under certain conditions from aqueous alkaline polymer solutions of unsubstituted cellulose carbamate through direct shaping according to the carbamate process.

Unmodified cellulose carbamate can be easily produced both according to the carbamate processes described in the prior art and also by any other carbamate processes (cf. e.g. DE 100 40 341, DE 196 35 473, DE 196 35 707, DE 102 53 672, DE 102 23 171, DE 102 23 174). Membranes can then be generated directly according to the carbamate process by cellulose carbamate having an average polymerisation degree DP of 180 to more than 500, preferably DP=200 to 400, with a degree of substitution DS of 0.1 to 0.6, preferably with a DS of 0.2 to 0.5 in a concentration of 5 to 12%, preferably 6 to 9%, being dissolved in aqueous sodium hydroxide solution of 4 to 12, preferably 5 to 8%, filtered and deaerated and then, directly after extrusion through hollow-core nozzles into acid or alkaline precipitation baths and the addition of a lumen-stabilising medium, being coagulated, predominantly with the addition of salt, and washed and after-treated in the standard manner, prepared using pore-preserving agents and dried. Decomposition stages can also be integrated into the process in order to adjust transport porosity and haemocompatibility. The use of solubilising additives in the dissolution of cellulose carbamate, such as zinc oxide and/or urea, is also possible, but not the preferred procedure since these substances would have to be removed again in the course of the process.

In forming the hollow membrane it is advantageous to use gases as the lumen-stabilising media which then no longer have to be expensively removed before being used in the artificial kidney, as is the case for example in the Cuoxam process, which uses liquid fatty acid esters as the lumen filler during the production process. The carbamate forming process is particularly suitable for using such inert gaseous lumen fillers as nitrogen or air for example and for metering e.g. by means of pressure-regulating systems, in order to set stable dimensions. A mixture with reactive gases can also be used which sometimes promotes the stability or structuring on the lumen side. Nevertheless it is naturally also possible to use inert or reactive liquid lumen fillers during hollow membrane manufacture according to the carbamate process, e.g. fatty acid esters, and to remove them then from the hollow membranes by using suitable solvents, e.g. ethanol. This procedure simplifies the maintenance of stable dimensions through forced feeding e.g. with gear-type pumps. Onicetan is an obvious preferred liquid lumen filler.

Flat membranes are also available. They can be produced in the conventional manner by drawing out the polymer solutions of cellulose carbamate with the aid of slit or ring dies and their one-sided and/or double-sided coagulation, application of the carbamate solution to rotating continuous belts, made e.g. of stainless steel, or simply by knifing with calibration knives, e.g. on glass surfaces etc., and similar devices with subsequent coagulation and/or decomposition and/or partial decomposition, washing, after-treating including preparing and drying, as used in principle in the hollow membrane manufacturing process.

After the solution has been shaped into hollow membranes or membranes, there follows coagulation in aqueous solutions of acids and/or inorganic salts, acid or alkaline salt solutions, possibly complete or above all partial decomposition in alkaline salt solutions, washing, preparation using pore-preserving agents and drying, both with relaxation or tension-free, with shrinkage impeded on one side or on both sides according to the generally known methods of contact, radiation or convection drying in moved and temperature-controlled gases. Coagulation in organic, e.g. alcoholic, precipitation baths is also possible.

The preferred type of membrane formation is hollow membrane formation. It comes about in the manner of a continuous wet forming process with circulating coagulation, decomposition, acidification, washing and after-treatment baths similar to the fibre formation in a continuous spinning process for textile man-made fibres, e.g. in the viscose spinning process. Forming, generally from the bottom to the top in a vertical spinning bath, takes place by regulated extrusion of the cellulose carbamate spinning solution through hollow-core nozzles into suitable coagulation baths with the defined addition of lumen-filling and lumen-stabilising media, the solidifying hollow fibre which is produced being taken off in a defined manner and conveyed through all the stages of the process e.g. also in stretching and relaxation steps up to drying. In this sense the other membrane-forming processes all proceed according to the same basic scheme.

As acid precipitation baths, those formed from sulphuric acid in a concentration of 1 to 250 g/l, preferably 30 to 140 g/l and a salts content of 0 to 350 g/l, preferably 80 to 280 g/l, have proved suitable, these salts coming preferably from the range of alkali salts such as sodium sulphate, sodium carbonate or even ammonium sulphate for example. But other acid/salt combinations are also suitable, such as sodium acetate or the practically salt-free acids on their own and also acid-free salt baths or soluble salts e.g. of zinc and aluminium with corresponding acids. Alkaline precipitation baths can cause a different type of precipitation if they are accompanied by salts such as sodium sulphate or sodium carbonate for example; sodium alkaline baths are preferred. The precipitation bath temperatures fluctuate in a range between −5° C. and roughly 50° C., preferably between 5° C. and 30° C.

Preferably, after coagulation and in an additional step, an at least partial decomposition of the membrane takes place. This can be accomplished by means of an alkaline decomposition bath, by isometric decomposition, by decomposition with partial relaxation or by decomposition through shrinkage.

Decomposition baths are alkaline baths of differing composition and temperature. Thus sodium hydroxide solutions of between 0.1 to 80 g/l together with salt contents of e.g. 0 to 250 g/l sodium sulphate are suitable, but sodium carbonate on its own also causes decomposition. Preferred decomposition baths are those which contain 1 to 60 g/l sodium hydroxide solution and 50 to 170 g/l sodium sulphate at application temperatures of 20 to 105° C., preferably 30 to 100° C. Solution concentration, temperature, salt content of the decomposition bath and treatment time of the fibre thus determine the rate of decomposition and the degree of decomposition of the cellulose carbamate hollow fibre. The degree of decomposition can if necessary go down to regenerated cellulose having a carbamate nitrogen content of less than 0.3%. An advantageous embodiment for matter transport is relaxing decomposition in a continuous and discontinuous process, the relaxation being able to go as far as complete shrinkage. In the continuous process, the relaxations are generally 0.1 to 10%, preferably 1 to 5%.

An important step in the formation of flat, tubular and hollow membranes is the intensive washing of the formed membranes either directly after coagulation or after an interposed decomposition stage which is possibly followed by acidification with e.g. diluted sulphuric acid. In the washing process, all the substances which are necessary for dissolving and shaping in the carbamate process are removed from the polymer body. In this way processing aids do not remain in the membranes and thus also do not reach the patient. What has proved successful is washing with distilled and/or demineralised water which has been purified over reverse osmosis membranes at elevated temperatures of up to roughly 80° C., preferably between 30 and 60° C.

As pore-preserving processing aids can be considered above all those in aqueous and/or alcoholic solution which are capable of penetrating into the initially moist porosity of the formed membrane. Inter alia glycerine and/or sorbitol have proved suitable for example, or also low molecular ethylene glycols or polyethylene glycols or substances from the group of sugar alcohols or mixtures thereof. Preferred, however, are aqueous or ethanol mixtures with glycerine and/or with glycerine and sorbitol. Pore-preserving agents of this type remain in the membrane during the production of the membrane separating equipment, e.g. artificial kidneys, and are only removed during the washing of membranes in preparation for use, in which they are removed e.g. with physiological salt solution and the membranes are simultaneously made to swell up. They have the task of making the transport porosity of the membranes available and keeping them permeable for the substances from the blood which have to be excreted through the urine. The amount and type of pore-preserving agents which remain in the membranes during drying determine the degree to which the membrane structure may swell again and thus the transport capacity. Mean concentration amounts in concentrations of up to approx. 40% in relation to cellulose or cellulose carbamate have proved to be suitable, preferably in the range between 5 and 20%. A conventional composition of a processing aid comprises an aqueous solution of 2.5% glycerine, 2.5% sorbitol and 2.5% polyethylene glycol (PEG) of relative molar mass 600.

Drying is largely tension-free at relatively low temperatures of roughly 20 to 50° C.; however temperatures outside this range are also perfectly possible. At high temperatures, the structure and/or the porosity can be fixed more strongly and if necessary the porosity can be limited. Isometric or partially relaxing drying is also possible. The type of drying can be used to influence the transport porosity. High relaxation and low temperatures intensify the transmembrane matter transport. The degree of relaxation can assume values of 0.2 to 8%. Complete shrinkage is dependent on the drying conditions and is to be aimed at.

The hollow membranes produced according to the carbamate process can be manufactured in a wide range of dimensions from an overall diameter of 180 to 500 μm. Dimensions above this are also possible. However, such dimensions are scarcely usual in artificial kidneys. Preferred dimensions are those of an outside diameter of 220 to 300 μm with wall thicknesses of 5 to 25 μm. Flat and tubular membranes with different dimensions of up to several hundred μm produced according to the carbamate process are also available. Preferred membrane thicknesses for blood detoxification are a wall thickness of 10 to 200 μm, especially 10 to 80 μm.

The invention will be explained and described further through the following examples.

Determining the analytical data of the spinning solutions and treatment baths proceeds customarily according to the methods known for the viscose spinning process (see e.g. K. Götze: Chemiefasern nach dem Viskoseverfahren [=“Chemical fibres produced according to the viscose process”], Berlin/Heidelberg/New York; Springer Verlag 1967). The ripening of cellulose carbamate spinning solutions can take place, as a modification of the standard manufacturing mode for viscose according to Hottenroth, by titrating the undiluted CC spinning solution with an aqueous sodium hydrogen carbonate solution. For determining the matter separation data of the membranes for dialysis and ultrafiltration, aqueous solutions of model substances of increasing relative molar mass such as urea and polyethylene glycols (PEG) of a defined relative molar mass were used. These methods for use with test dialysers and the appropriate relative molar masses of the test substances have been described and are not repeated again here (Gensrich, J., Scholz, C., Holtz, M., Müller, K. and Paul, D.: Hohlmembranen nach dem Viskoseverfahren—vergleichende Charakterisierung [=“Hollow membranes produced according to the viscose process—comparative characterisation”], 3^{rd} Technical Conference “Theorie und Praxis der Membrantrennprozesse” [=“Theory and practice of membrane separation processes”] 25-26 Oct. 1988; Conference record: Scientific contributions from the School of Engineering Köthen 4 (1988), 88-108). The matter separation properties of flat and hollow membranes are available according to the same principles in a pressure or two-cell apparatus. Complement activation with the aid of Factor C3 des Arg-ELISA was determined by the enzyme immunoassay method and the values obtained were compared in percentage terms with those for regenerated cellulose (membranes produced according to the Cuoxam process). (The measured values for regenerated cellulose according to the Cuoxam process are made equal to 100%.)

### EXAMPLE 1

In a cooled mixing vessel, a polymer solution in an aqueous sodium hydroxide solution was produced at 0° C. from a cellulose carbamate (CC) having a carbamate nitrogen content (N) of 2.9% and a DP_{cuoxam} of 280, such that a composition of 8.4% cellulose carbamate and 7.3% NaOH was produced which, after the conventional filtration and vacuum deaeration for polymer solutions as well as a typical time and temperature-dependent, nitrogen- degrading ripening for carbamate solutions in alkali media, had a ripeness of 14° H (Hottenroth degree, based on the viscose ripeness). This so-called spinning solution (CL) was drawn out with a Wasag ruler having a gap width of 0.3 mm to form four membranes (M) on a glass slab; membrane 1 was precipitated with an aqueous ammonium sulphate solution of 264 g/l, membrane 2 with an aqueous bath comprising 80 g/l sulphuric acid and 140 g/l sodium sulphate, membrane 3 with an aqueous precipitation bath comprising 20 g/l ethanoic acid and the 4^{th} membrane was coagulated with an aqueous precipitation bath of 5 g/l sodium hydroxide solution and 100 g/l sodium sulphate at room temperature. All these membranes were after-treated in a uniform way: membrane decomposition in an aqueous sodium carbonate bath of 50 g/l at 60° C. to a carbamate nitrogen content of 1.5%, washed with de-ionised water, washed to form the pure cellulose carbamate membrane and clamped initially moist in a pressure apparatus and, by applying a pressure of 500 Torr, the pure water permeability (PWP) was measured. A set of identical membranes M5 to M8 was placed, after identical manufacture to that of M1 to M4, in a 10% aqueous glycerine solution and dried in air at 30° C. Before the PWP was determined, M5 to M8 were washed free of glycerine with distilled water. The following PWP values were measured in ml/hour per m and per kPa:

| | Moist membrane | PWP |
|---|---|---|
| Membrane | strength [μm] | [ml/h² · kPa] |
| M1 | 35 | 25 |
| M2 | 32 | 28 |
| M3 | 37 | 22 |
| M4 | 33 | 20 |
| M5 | 31 | 20 |
| M6 | 28 | 23 |
| M7 | 29 | 18 |
| M8 | 30 | 17 |

After being washed with physiological salt solution, membranes M6 cause a complement activation of 61% with a carbamate nitrogen content of 1.5%.

### EXAMPLE 2

Extrusion took place from the same solution as in Example 1, after diluting the spinning solution with 7.3% aqueous sodium hydroxide solution to 7.3% CC content in a defined continuous operation, by extruding this solution by means of conventional gear-type pumps with a ripeness of 19° H and a viscosity of 3 Pas through a hollow-core nozzle (outside diameter of the annular gap 540 μm, slit width 120 μm) into an aqueous precipitation bath of 80 g/l sulphuric acid and 260 g/l ammonium sulphate at 16° C., an internal excess pressure above the hydrostatic pressure of the vertical precipitation bath of nitrogen was applied to the lumen through the hollow-core nozzle such that, after all the treatment stages, the hollow membranes in the dry state had an inside diameter of 223 μm and a wall thickness of 12.4 μm. The treatment stages comprised, after coagulation from the bottom to the top in the vertical spinning bath, drawing-out between the nozzle and godet 1 and stretching in air by 40% between godets 1 and 2, decomposition in a bath of 20 g/l sodium hydroxide solution and 110 g/l sodium sulphate at 97° C. on godet 2, washing this hollow fibre with diluted sulphuric acid and de-ionised water on washing rollers, preparing it with an aqueous solution of 2.5% each of glycerine, sorbitol and PEG600 and drying it isometrically at 50° C. In the decomposition stage, the hollow fibre is relaxed by 1.5%. After embedding in a test dialyser, swelling the fibres with water and washing away the processing aid, the hollow membranes had the following performance data by comparison with aqueous solutions of test substances:

Permeability coefficient for urea P_{Urea}*10⁻³=34 cm/min; a PWP for water of 47 ml/h*m²*kPa and a cut-off_{PEG} of 13000 Dalton. After washing with physiological salt solution, the hollow membranes had a complement activation of 52% with a carbamate nitrogen content of 0.5%.

### EXAMPLE 3

The hollow membranes, manufactured according to Example 2, produce the following values without a decomposition stage:

| | |
|---|---|
| Spinning ripeness [° H]: | 14 |
| Inside diameter [μm]: | 211 |
| Wall thickness [μm]: | 12.4 |
| N [%]: | 1.5 |
| PWP [ml/h*m²*kPa]: | 48 |
| P_{Urea} [cm/min]: | 39 |
| P_{PEG1383} [cm/min]: | 3.2 |
| Selectivity for PEG 6110 [%]: | 67 |

### EXAMPLE 4

From an undiluted spinning solution as per Example 1, in the same operation as in Example 2 but with a decomposition bath temperature of 82° C., hollow membranes were produced which had the following properties:

| | |
|---|---|
| Spinning ripeness [° H]: | 11.4 |
| Inside diameter [μm]: | 223 |
| Wall thickness [μm]: | 11.4 |
| N [%]: | 0.8 |
| PWP [ml/h*m²*kPa]: | 45.6 |
| P_{Urea} [cm/min]: | 30.3 |
| Selectivity for PEG 6110 [%]: | 71 |

### EXAMPLE 5

From a CC with an N of 3.4% and a DP of 296, a spinning solution was manufactured in the manner of Example 1 with a CC content of 7.5% and an alkali content of 8.0%. It was shaped into hollow membranes in an aqueous precipitation bath of 79 g/l sulphuric acid, 264 g/l ammonium sulphate and 33 g/l sodium sulphate at 36° C. and a stretching bath at 34° C., comprising 98 g/l sulphuric acid, 11 g/l sodium sulphate and 7 g/l ammonium sulphate, with stretching of 5% and a nozzle distortion of 1.42 in the manner of Example 2 but without a decomposition bath, and the properties were determined:

| | |
|---|---|
| Spinning ripeness [° H]: | 6.5 |
| Inside diameter [μm]: | 301 |
| Wall thickness [μm]: | 26.6 |
| N [%]: | 0.77 |
| (Average degree of substitution DS approx. 0.1) | |
| PWP [ml/h*m²*kPa]: | 76 |
| P_{Urea} [cm/min]: | 19.6 |
| P_{PEG1383} [cm/min]: | 3.3 |
| Selectivity for PEG 6110 [%]: | 46 |

These hollow membranes triggered a complement activation of 46%. They could be washed away perfectly from the human blood used by means of physiological salt solution.

### EXAMPLE 6

From a spinning solution, produced according to the procedure as per Example 1 from a CC (N=2.8%) with a DP of 290, having the composition 9.1% CC and 7.2% sodium hydroxide solution as described in Example 2, with a ripeness of 11.9° H, in an aqueous precipitation bath of 105 g/l sulphuric acid and 140 g/l sodium sulphate at 15° C. and an aqueous decomposition bath of 16 g/l sodium hydroxide solution and 76 g/l sodium sulphate at 59° C. (Fibre 1) or 82° C. (Fibre 2), hollow fibres with the following properties were produced:

| Hollow fibre | 1 | 2 |
|---|---|---|
| Inside diameter [μm]: | 225 | 222 |
| Wall thickness [μm]: | 13.3 | 14.2 |
| N [%]: | 1.5 | 1.2 |
| PWP [ml/h*m²*kPa]: | 22 | 26 |
| P_{Urea} [cm/min]: | 21.5 | 26.0 |
| Selectivity for PEG 6110 [%]: | 74 | 75 |

### EXAMPLE 7

To produce a spinning solution (CC=8.4%; NaOH=7.3%), recourse was had to a CC with N=2.9 and DP=286, which was dissolved as described in Example 1. This solution was spun into hollow fibres at a ripeness of 15° H as described in Example 2, but with a precipitation bath of 80 g/l sulphuric acid, 262 g/l ammonium sulphate at 17° C. and a decomposition bath of 19 g/l sodium hydroxide solution and 108 g/l sodium sulphate at 81° C. with stretching of 60% and a final take-off of 20.1 m/min. The hollow membranes had the following properties:

| | |
|---|---|
| Inside diameter [μm]: | 210 |
| Wall thickness [μm]: | 10.3 |
| N [%]: | 0.7 |
| PWP [ml/h*m²*kPa]: | 34 |
| P_{Urea} [cm/min]: | 36.5 |
| Selectivity for PEG 6110 [%]: | 79 |

### EXAMPLE 8

Hollow fibres were produced as described in Example 7 with the difference that the ripeness was 10° H, the stretching 40% and the final take-off 26.5 m/min. The hollow membranes had the following properties.

| | |
|---|---|
| Inside diameter [μm]: | 200 |
| Wall thickness [μm]: | 8.3 |
| N [%]: | 0.8 |
| PWP [ml/h*m²*kPa]: | 50.2 |
| P_{Urea} [cm/min]: | 36.0 |
| Selectivity for PEG 6110 [%]: | 74.8 |

### EXAMPLE 9

From a further CC with a DP of 212 and an N content of 2.6%, an aqueous alkaline spinning solution was produced according to the procedure in Example 1, which had a composition of 8.4% cellulose and 7.3% alkali. Two hollow membranes were formed from this by precipitation into an acid precipitation bath (79 g/l sulphuric acid; 330 g/l ammonium sulphate) and partial decomposition in an aqueous decomposition bath at 95° C., comprising 21 g/l sodium hydroxide solution and 110 g/l sodium sulphate, according to the sequence in Example 2. Three different hollow fibres were produced. Some of hollow fibres 2 were, after preparation, taken up in a bundle as hollow fibres 3, initially damp, onto a reel, completely relaxed and dried at room temperature without tension, shrinking freely. The differing test conditions and the properties achieved are as follows:

| Hollow fibre | 1 | 2 | 3 |
|---|---|---|---|
| Precipitation bath temperature [° C.]: | 15 | 30 | 30 |
| Ripeness [° H]: | 15 | 15 | 15 |
| Inside diameter [μm]: | 223 | 227 | 230 |
| Wall thickness [μm]: | 11.4 | 12.4 | 13.1 |
| N [%]: | 0.8 | 0.9 | 0.8 |
| PWP [ml/h*m²*kPa]: | 100.2 | 56.6 | 89.3 |
| P_{Urea} [cm/min]: | 20.4 | 30.3 | 36.0 |
| Selectivity for PEG 6110 [%]: | 46.6 | 50.2 | 45.3 |

### EXAMPLE 10

Under the same conditions as Example 9, additional hollow membranes were produced in further tests. The differing test conditions and the properties achieved emerge as follows:

| Hollow fibre | 1 | 2 | 3 | 4 |
|---|---|---|---|---|
| Precipitation bath temperature [° C.]: | 15 | 30 | 15 | 30 |
| Ripeness [° H]: | 10.3 | 10.3 | 7.5 | 7.5 |
| Inside diameter [μm]: | 220 | 223 | 208 | 210 |
| Wall thickness [μm]: | 12.4 | 12.4 | 12.9 | 12.4 |
| N [%]: | 0.6 | 0.6 | 0.4 | 0.4 |
| PWP [ml/h*m²*kPa]: | 71.3 | 66.5 | 71.2 | 57.8 |
| P_{Urea} [cm/min]: | 33.7 | 35.9 | 35.4 | 32.2 |
| Selectivity for PEG 6110 [%]: | 53.6 | 56.7 | 54.2 | 69.9 |

### EXAMPLE 11

Under the same conditions as Example 9, additional hollow membranes were produced in further tests. The differing test conditions and the properties achieved are listed below:

| Hollow fibre | 1 | 2 | 3 |
|---|---|---|---|
| Sulphuric acid in the precipitation bath [g/l]: | 40.2 | 40.2 | 119.0 |
| Ammonium sulphate in the precipitation bath [g/l]: | 268 | 268 | 268 |
| Precipitation bath temperature [° C.]: | 15.5 | 30 | 16 |
| Ripeness [° H]: | 14.3 | 14.3 | 14.5 |
| Inside diameter [μm]: | 212 | 256 | 243 |
| Wall thickness [μm]: | 12.4 | 10.3 | 10.3 |
| N [%]: | 0.8 | 0.9 | 1.0 |
| PWP [ml/h*m²*kPa]: | 63.7 | 96.1 | 64.2 |
| P_{Urea} [cm/min]: | 38.3 | 35.3 | 28.8 |
| P_{PEG1383} [cm/min]: | 3.4 | 4.3 | 3.1 |
| Selectivity for PEG 6110 [%]: | 62.5 | 54.1 | 64.5 |

### EXAMPLE 12

Under the conditions of Example 9, additional hollow membranes were produced in further tests with different spinning solution compositions. The differing test conditions and the properties achieved are listed below:

| Hollow fibre | 1 | 2 | 3 |
|---|---|---|---|
| Cellulose in the spinning solution [%]: | 7.2 | 7.2 | 7.2 |
| Sodium hydroxide solution in the spinning solution: | 8.3 | 6.3 | 6.3 |
| Sulphuric acid in the precipitation bath [g/l]: | 80 | 80 | 80 |
| Ammonium sulphate in the precipitation bath [g/l]: | 339 | 339 | 339 |
| Precipitation bath temperature [° C.]: | 15 | 15 | 30 |
| Ripeness [° H]: | 14.8 | 14.1 | 14.1 |
| Inside diameter [μm]: | 220 | 246 | 257 |
| Wall thickness [μm]: | 14.5 | 12.4 | 10.3 |
| N [%]: | 1.0 | 0.9 | 0.8 |
| PWP [ml/h*m²*kPa]: | 104.2 | 108.6 | 107.2 |
| P_{Urea} [cm/min]: | 35.5 | 37.5 | 35.1 |
| P_{PEG1383} [cm/min]: | 4.9 | 4.3 | 4.7 |
| Selectivity for PEG 6110 [%]: | 53.7 | 45.8 | 51.2 |

### EXAMPLE 13

A cellulose carbamate solution produced according to Example 1 was shaped into a hollow fibre according to the sequence in Example 2 in an aqueous alkaline precipitation bath comprising 10 g/l sodium hydroxide solution and 221 g/l sodium sulphate with a ripeness of 8.5° H, Onicetan, a fatty acid ester, being added by means of a gear-type pump. The stretching between godets 1 and 2 in air is 20%, the final take-off 15.3 n/min. After coagulation, the hollow fibre passes through an acidification bath at room temperature, comprising 40 g sulphuric acid per litre of water, in order then to run, without a decomposition bath, directly into the washing path of distilled water at 40° C. and to be after-treated as described in Example 2. Drying took place with relaxation of 3%. A second portion of the spinning solution was precipitated, exchanging the precipitation bath for ethanoic acid, into a precipitation bath of 200 g ethanoic acid per litre water without a preceding acidification path, again directly into the decomposition path and was treated, acidified, washed, prepared and dried as per the sequence in Example 2, and a third portion of the solution was shaped into hollow membranes in the same way as the second portion of the spinning solution with the addition of ethanol to the acetous precipitation bath (15 g/l ethanol in the precipitation bath). After these hollow fibres had been embedded in the test dialyser, they were washed free of Onicetan using ethanol and freed of the ethanol using distilled water. These hollow membranes had the following properties:

| Hollow fibre | 1 | 2 | 3 |
|---|---|---|---|
| Precipitation bath | alkaline | acetous | acetous with |
| | | | ethanol |
| Precipitation bath temperature [° C.]: | 28 | 23 | 25 |
| Inside diameter [μm]: | 238 | 240 | 230 |
| Wall thickness [μm]: | 12.9 | 12.0 | 12.4 |
| N [%]: | 1.3 | 0.5 | 0.6 |
| PWP [ml/h*m²*kPa]: | 24 | 25 | 19 |
| P_{Urea} [cm/min]: | 26 | 25 | 30 |
| Selectivity for PEG 6110 [%]: | 75 | 72 | 81 |
| Complement activation [%]: | 63 | — | — |

### EXAMPLE 14

From a cellulose carbamate, produced according to DE 102 23 174 and washed residue-free with diluted ethanoic acid and distilled water, with a DP_{Cuoxam} of 255 and an N of 3.7%, hollow membranes were produced using gaseous nitrogen with the addition of approx. 2% by volume SO3₃ as the lumen filler. The cellulose carbamate spinning solution produced according to Example 1, having a CC content of 8.8% and an NaOH content of 7.2%, had, after filtration and deaeration, a spinning ripeness of 12.5° H. It was shaped into hollow membranes in an acid aqueous spinning bath at 10° C. with a content of 79 g sulphuric acid and 288 g ammonium sulphate per litre of aqueous spinning bath. The decomposition process was carried out with a decomposition bath at 60° C. having a sodium sulphate content of 120 g/l and increasing sodium hydroxide solution contents of 5 g/l (hollow fibre 1), 18 g/l (hollow fibre 2) and 36 g/l (hollow fibre 3), and after-treated as described in Example 2. The hollow membranes had the essential properties listed below:

| Hollow fibre | 1 | 2 | 3 |
|---|---|---|---|
| NaOH in the decomposition bath [g/l]: | 5 | 18 | 36 |
| Inside diameter [μm]: | 240 | 246 | 238 |
| Wall thickness [μm]: | 11.7 | 11.5 | 11.2 |
| N [%]: | 1.2 | 0.8 | 0.4 |
| PWP [ml/h*m²*kPa]: | 64 | 69 | 74 |
| P_{Urea} [cm/min]: | 29 | 34 | 33 |
| Selectivity for PEG 6110 [%]: | 62 | 60 | 58 |
| Complement activation [%] | 67 | 55 | 58 |

After being washed free of the human blood used by means of physiological salt solution, the hollow membranes were completely colourless and residue-free.

1. Method for manufacturing membranes for haemodialysis, haemofiltration and/or plasmapheresis, comprising the following method steps:
a) dissolving cellulose carbamate having a carbamate nitrogen content of 1 to 6% in sodium hydroxide solution, followed by shaping into a membrane,
b) coagulation with acid or alkaline solutions,
c) washing the membrane,
d) finishing the membrane by adding a pore-preserving processing aid which penetrates into the pores of the membrane,
e) drying the membrane.

2. Method according to claim 1, characterized in that the cellulose carbamate has a DP of 180 to 550, especially 200 to 400, and a DS of 0.1 to 0.6, especially 0.2 to 0.5.

3. Method according to claim 1, characterized in that in b) aqueous or alcoholic solutions are used.

4. Method according to claim 1, characterized in that in b) the acid or alkaline aqueous solutions contain inorganic salts.

5. Method according to claim 1, characterized in that, after coagulation, in an additional step, an at least partial decomposition of the membrane takes place in an alkaline decomposition bath, by isometric decomposition, decomposition with partial relaxation or by decomposition through shrinkage.

6. Method according to claim 5, characterized in that treatment with an acid solution takes place after the decomposition.

7. Method according to claim 1, characterized in that glycerin, sorbitol, low-molecular ethylene glycols or polyethylene glycols, sugar alcohols and or mixtures thereof, all contained in aqueous and/or alcoholic solutions, are used as pore-preserving processing aids.

8. Method according to claim 7, characterized in that the processing aid comprises an aqueous solution having 2.5 wt-% glycerin, 2.5 wt-% sorbitol and 2.5 wt-% polyethylene glycol (PEG600).

9. Method for manufacturing hollow membranes according to claim 1, characterized in that in step b) the coagulation takes place with the addition of a lumen-stabilizing gaseous or liquid medium.

10. Method according to claim 9, characterized in that inert air and nitrogen is used as the lumen-stabilizing gaseous medium.

11. Method according to claim 9, characterized in that the hollow membrane is produced continuously via a wet forming process.

12. Method for manufacturing tubular or flat membranes according to claim 1, characterized in that in a) the solution is drawn out by means of slit dies or ring dies.

13. Method for manufacturing tubular or flat membranes according to claim 1, characterized in that in a) the solution is applied to rotating continuous belts.

14. Method for manufacturing tubular or flat membranes according to claim 1, characterized in that in a) the solution is drawn on a surface by means of calibration knives.

15. Method for manufacturing tubular or flat membranes according to claim 1, characterized in that in e) the drying takes place under tension.

16. Method for manufacturing tubular or flat membranes according to claim 1, characterized in that in e) the drying takes place with partial relaxation.

17. Method for manufacturing tubular or flat membranes according to claim 1, characterized in that in e) the drying takes place with shrinkage.

18. Membrane for haemodialysis, haemofiltration and/or plasmapheresis formed from substituted or unsubstituted cellulose carbamate having a carbamate nitrogen content in the range between 0.1 and 6%.

19. Membrane according to claim 18, characterized in that the carbamate nitrogen content is in the range between 0.2 and 4%.

20. Membrane according to claim 18, characterized in that the membrane is a hollow, tubular or flat membrane.

21. Membrane according to claim 20, characterized in that the hollow membranes have an outside diameter of 180 to 500 μm, especially 220 to 300 μm.

22. Membrane according to claim 20, characterized in that the wall thickness of the hollow membranes is between 5 and 25 μm.

23. Membrane according to claim 20, characterized in that the tubular or flat membranes have a wall thickness of 10 to 200 μm, especially 10 to 80 μm.

24. Membrane according to claim 18, characterized in that it can be manufactured using the method according to one of claims 1 to 8.

25. Hollow membrane according to claim 18, characterized in that it can be manufactured using the method according to one of claims 9 to 11.

26. Tubular or flat membrane according to claim 18, characterized in that it can be manufactured using a method comprising drawing out solution using slit dies or ring dies.

27. A method of using the membranes according to claim 18 for the detoxification of blood by haemodialysis, comprising employing said membrane in high flux dialysis and/or haemofiltration.

28. A method according to claim 27 for the ultrafiltration, haemodiafiltration and plasmapheresis of the blood.

29. A method according to claim 27 performed in artificial kidneys.

30. A method according to claim 27 performed in ultrafiltration equipment in the form of hollow-fiber dialysers.
